(19) United States
(12) Patent Application Publication
(10) Pub. No.: US 2017/0356834 A1
(43) Pub. Date: Dec. 14, 2017

(54) METHOD FOR QUANTITATIVELY AND QUALITATIVELY DETECTING PARTICLES IN LIQUID

(71) Applicant: Grundfos Holding A/S, Bjerringboro (DK)

(72) Inventors: Kåre IVERSEN, Langå (DK); Christian GULDBÆK SMITH, Skødstrup (DK); Mathis DAHLQVIST, Vejle (DK)

(73) Assignee: Grundfos Holding A/S, Bjemingbro (DK)

(21) Appl. No.: 15/538,430

(22) PCT Filed: Nov. 30, 2015
(86) PCT No.: PCT/EP2015/078110

§ 371 Date: Jun. 21, 2017

(30) Foreign Application Priority Data
Dec. 22, 2014 (EP) 14199823.7

(51) Int. Cl. G01N 15/02; G06K 9/00; G01N 21/958; G01N 33/18; G06K 9/40; G01N 15/14; G01N 21/88; G01N 21/15; G01N 15/00

(52) U.S. Cl. CPC G01N 15/0227; G01N 21/8851; G06K 9/0014; G01N 21/958; G01N 21/15; G01N 33/1893; G06K 9/40; G01N 15/1475; G01N 2015/0088; G01N 2021/8887; G01N 2021/157; G01N 2015/1493

(57) ABSTRACT

A method for the quantitative and/or qualitative detection of particles in fluid, with which the fluid to be examined is introduced into a beam path of an optical device, between at least one light source and the image acquisition sensor with a matrix of light-sensitive cells. Pixel values of the cells are detected and the distribution of the pixel values is at least partly determined. The pixel value or values which have been determined most often are used as a value or average value for a background signal. A signal is outputted or the method is interrupted, on reaching a maximal permissible value for the background signal.

### CROSS REFERENCE TO RELATED APPLICATIONS

This application is a United States National Phase Application of International Application PCT/EP2015/078110, filed Nov. 30, 2015, and claims the benefit of priority under 35 U.S.C. §119 of European Application 14199823.7, filed Dec. 22, 2014, the entire contents of which are incorporated herein by reference.

### FIELD OF THE INVENTION

The invention relates to a method for detecting particles in fluid, with which the fluid to be examined is introduced into a beam path of an optical device, between at least one light source and the image acquisition sensor with a matrix of light-sensitive cells, with which the pixel values of the cells are detected and the distribution of the pixel values is at least partly determined.

### BACKGROUND OF THE INVENTION

Methods for the optical detection of particles in a fluid are counted as belonging to the state of the art. EP 2 469 264 A1, WO 2010/063293 A1 and WO 2014/094790 A1 are referred to in this context, and with these disclosures, the detection is carried out by way of an image acquisition sensor with a matrix of light-sensitive cells, for example of a CCD sensor, with which the fluid to be examined is introduced into the beam path of an optical device, between at least one light source and the image acquisition sensor. These devices, although being envisaged for the qualitative and quantitative detection of particles in a fluid in the stationary condition, however are also used for quasi stationary, continuous examination, for example for the regular checking of drinking water. As a rule, they have a sample carrier for this, whose fluid is exchangeable. Suitable valves, as the case may be pumps and a procedural control are provided for this.

With the evaluation of the signal of the image acquisition sensor (imaging sensor), it is counted as belonging to the state of the art to detect the pixel values of the individual cells and to at least partly determine the statistical distribution of the pixel values. The pixel value or the pixel values which are thereby determined most often are used as a value or average value for a background signal which is subtracted from the actual pixel value with a subsequent signal processing, in order in this manner to numerically fade out the background or the background noise and to simplify and improve the particle detection. Kim, Seongjai and Lim, Hyeona “Method of Background Subtraction for Medical Image Segmentation” in Nature 2013 is referred to in this context.

It is particularly with the examination of drinking water, but also with other fluids which encourage an accumulation of deposits or organic growth such as biofilm, that the long-term stability of the sample carrier, in particular of the sample carrier window is a problem. The deposits do not accumulate suddenly, but continuously and this being the case at a speed which cannot be foreseen, and lead to the fact that the results become less and less accurate in the course of time up to the complete failure of the measurement. It is therefore necessary for the sample carrier, i.e. the component, in which the fluid is led along a window arranged in the beam path of the optical device, to be exchanged at regular intervals. The exchange of the sample carrier must be effected in comparatively short intervals as a precautionary measure since the speed of the increase of the deposits or accumulations varies, and this is cumbersome and expensive.

### SUMMARY OF THE INVENTION

Against this background, it is an object of the invention, to provide a method which detects these deposits and this, if possible, being with already existing means.

With the method according to the invention for the quantitative and/or qualitative detection of particles in fluid, the fluid to be examined is arranged in a beam path of an optical device, between at least one light source and an image acquisition sensor with a matrix of light-sensitive cells. Thereby, the pixel values of the cells are detected and the distribution of the pixel values is at least partly determined, wherein the pixel value or pixel values which have been determined most often, are used as a value or average value for a background signal, as is basically counted as belonging to the state of the art. According to the invention however, a signal is outputted or the detection method is interrupted on reaching a predefined limit value for the background signal.

The basic concept of the method according to the invention is to evaluate the value for the background signal and which per se is to be determined in any case, as to whether this value lies below or above a predefined limit value for the background signal, wherein a signal is outputted or the method is interrupted on reaching this limit value or on falling short of it. Thus a value which is to be determined in any case is determined, in order to ascertain whether an unallowably high degree of deposits or contamination of the examination window but also of the remaining optics between the light source and sensor is present, in order to either output a signal which signalizes the reaching or exceeding of this limit value, or to interrupt the detection method. Thereby, if the limit value is specified or set at a sufficient distance to a possible maximally allowable value, a signal output which indicates to the user that the sample carrier is then to be shortly exchanged or at least cleaned or sets an automatic control in action for this can be sufficient.

The method according to the invention for the quantitative and/or qualitative detection of particles in fluid is basically independent of whether the fluid is still or flows. The method according to the invention however is preferably applied to quasi stationary fluids, which means the fluid to be examined is allowed to flow in a sample carrier, whereupon the feed and discharge are however closed, and a certain time is waited, until the particles located in the fluid no longer move, which means suspended particles have reached a quasi stationary condition, and, with particles which are significantly heavier or lighter than the fluid, these have either completely sunk to the bottom or floated to the top.

The method according to the invention has the advantage that it utilizes present resources and can be implemented quasi with regard to software, which means it creates no additional costs.

Ideally thereby, the distribution of the pixel values of the complete matrix of light-sensitive cells is determined. It is to be understood that it can also be sufficient if only a large part of this matrix is evaluated, if e.g. a part of the cells are to be used for other purposes and is not available for this. Thereby, it does not necessarily need to be a pixel value which has been determined most often, but it can also be several pixel values. Usually, a peak results in a distribution diagram, and this peak is then used as a value or an average value for a background signal, for example by way of forming a mean. This background signal which is also indicated as a noise signal is deducted from each individual pixel value with the later evaluation of the pixel values, in order to thus ensure a quasi noise-free evaluation.

Thereby, according to an advantageous further development of the invention, a signal is outputted on reaching a first predefined limit value for the background signal, and the detection method is interrupted on reaching a second predefined limit value. Such a two-stage solution makes sense, in order, in good time, to indicate an increasing contamination of the optical device, in particular of the sample carrier window and, in a further step, at the latest when the contamination is so great that a statistically valid evaluation is no longer possible, to stop the detection method. The limit values are thereby usefully to be predefined such that the first limit value lies significantly below the permissible limit, within which a reliable quantitative and preferably also qualitative detection of particles in the fluid is ensured, and the second limit value lies just below this limit value or is formed by the limit value itself, so that on reaching this limit value, it is ensured that a detection is no longer effected or the evaluation is aborted.

With the method according to the invention, advantageously a multitude of images is recorded in different image planes, such as is described for example in WO 2014/094790 A1. It is useful to form the background signal by way of the mean of values or average values of several successively recorded images, preferably of 500 to 1500 images of different image planes, but of the same fluid sample, in order to carry out a statistically valid evaluation with regard to the background signal.

According to an advantageous further development of the method according to the invention, one envisages the values for the background signal of temporally successively effected detections of particles in fluid being registered and a change speed being determined by way of the temporal change of the registered values, in order to not only ensure the quality of the current examination, but in particular to detect and to evaluate the increase of deposits or contamination within the optical device, in particular of the sample carrier window. Then, on account of this change speed, one can determine without further ado, as to how long the detection process can still be continued without an intervention in the system being necessary, be it for cleaning purposes or for the exchange of components. Thereby, the change speed gives an indication as to the increase of deposits or contamination which is to be reckoned with, and within which time. The evaluation of the change speed also permits the early recognition as to when unexpected and sudden changes occur within the optical system.

According to an advantageous further development of the method according to the invention, the registration of these values for the background signal and the evaluation of the temporal change of the registered values can advantageously be used to determine when a predefined limit value for the background signal is likely to be reached. A correspondingly designed device, in a suitable display can thus specify when a cleaning of the device or an exchange of the sample carrier is likely to have to be effected at the latest.

The background signal is thus usefully used as a measure for the contamination in the beam path, and a fluid carrier or sample carrier which receives the fluid to be examined is cleaned or exchanged which means is replaced by a new or cleaned fluid carrier, on reaching a predefined limit value.

A constant examination of this background signal is basically not necessary, since the contamination in such optical devices in particular the biofilm formation within the sample carrier window which is being discussed here, is not effected abruptly, but over several days or weeks. According to the invention, the determining of the background signal is however advantageously effected before each detection of particles, or at least in predefined time intervals, in order to ensure adequate system reliability. According to an advantageous development of the invention, one can basically also envisage the contamination degree of the sample carrier being determined in the unfilled condition, i.e. determining the background signal when the sample carrier is not yet filled with fluid, or emptying and preferably also drying this before the measurement procedure.

Thereby, according to an advantageous further development of the invention, on evaluating the pixel values of the cell matrix of the sensor, this matrix can be divided into a multitude of preferably equally large part-matrices, and a background signal formed for each part-matrix. Thereby, the part-matrix or the part-matrices, whose background signal exceeds a predefined further limit value, is excluded from the further valuation for detecting the particles. This further development according to the invention has the advantage that individual, greatly contaminated parts of the sample carrier window can be excluded in a targeted manner with the detection, and this also includes larger particles, such as air bubbles or particles in this size magnitude, if for example it is a case of the detection of particles which are significantly smaller such as bacteria or turbidities for example.

The distribution of the pixel values can not only be used for determining the background signal, but with this advantageously the particles are also categorized with regard to size. In particular, such a categorization with regard to size can already be a first selection if it is a question of the detection of certain particles, and this with regard to the method and also evaluation technology can be carried out in a relatively simple manner.

The evaluation of the pixel values is usefully effected digitally. It has also be found to be useful to specify the limit value for the background signal and/or the further limit value for the background signal of a part-matrix at 10 to 20% of a maximal pixel value. This percentage specification is independent of the resolution of the pixel value. With an 8-bit resolution of a pixel value, which corresponds to a resolution of 256 brightness steps, it has been found to be advantageous to fix the limit value for the background signal between 30 and 50, preferably between 35 and 40. This means that with the specified resolution, after deduction of the background signal, at all events more than 200 different brightness steps still remain. Thereby, this limit value is independent of whether it is the case of an optical device, with which the light source directly beams through the sample carrier window (brightfield technique), or whether it is the case, with which only indirect light reaches the sample carrier (darkfield technique). What is essential is the fact that a measurement range of more than 200 steps remains in each case.

With an 8-bit resolution, a value between 35 and 45 has been found to be advantageous as a further limit value for the background signal of a part-matrix. Thus with the method according to the invention, then only the part-matrices which have a background signal of the part-matrix which exceeds a value between 35 and 45, depending on which value has been predefined here, are excluded from the further detection procedure.

The device according to the invention for the optical detection of particles in a fluid comprises a light source, a image acquisition sensor with a matrix of light-sensitive cells and a fluid carrier which is arranged in the beam path therebetween, as well as control and evaluation electronics which detects the pixel values of the cells and at least partly determines the distribution of the pixel values and uses the pixel value or pixel values which have been determined most often, as a value or average value for a background signal. According to the invention, the control and evaluation electronics are designed such that a signal is outputted or the detection procedure is interrupted, on reaching a predefined limit value for the background signal. In an analogous manner, the control and evaluation electronics according to the invention can be designed, in order to also carry out the previously described further method features.

Parts of the invention are hereinafter explained by way of representations. The present invention is described in detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a diagram, which on the one hand shows the course of the background signal over a time period of more than a year and on the other hand the frequency distributions of the pixel values for this time period;

FIG. 2a is an image of a sample which is detected by way of a CCD sensor;

FIG. 2b is an image of the frequency distribution of the pixel values of the image according to FIG. 2a;

FIG. 3a is a further image according to FIG. 2a, with an increased deposit formation;

FIG. 3b is an image of the frequency distribution of the pixel values of the image according to FIG. 3a;

FIG. 4a is, in a greatly simplified representation, an image with large and small particles;

FIG. 4b is an image showing how the large particles of the image 4a are faded out with the evaluation;

FIG. 5a is a further image corresponding to FIG. 4a; and

FIG. 5b is an image of the respective fading-outs with the evaluation.

### DESCRIPTION OF THE PREFERRED EMBODIMENTS

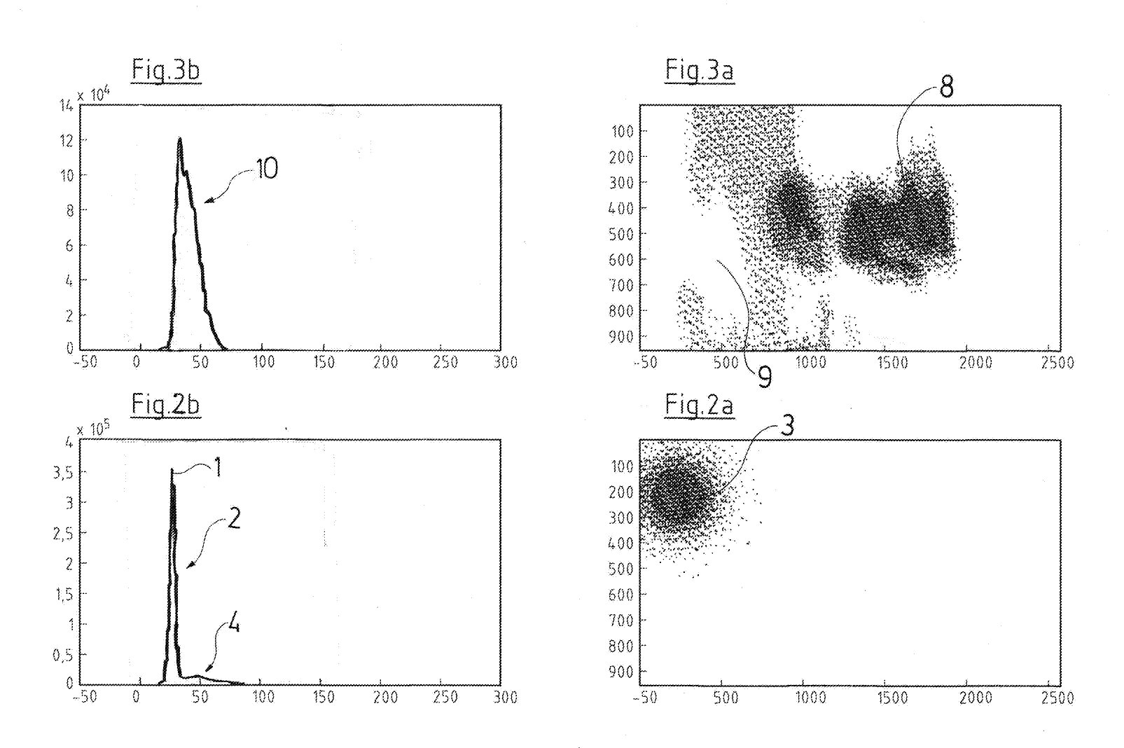

Referring to the drawings, a device which is known per se is used with the quantitative and qualitative detection of particles in a fluid, with which a sample carrier with a window is arranged between a light source and a CCD sensor. Thereby, the illumination of the window can either be effected in a direct manner (brightfield technique) or in an indirect manner (darkfield technique), which however is of no significance for the methods which are being discussed here. The representations according to FIG. 2a to 5a, as is useful for examining drinking water, are taken in the darkfield technique, but are represented in an inverted manner, in particular in order to ensure the replication and the optical recognizability. Indeed, the detected particles in the darkfield technique appear white against a black or grey background. A value for a background signal is firstly determined before the actual signal evaluation of the sensor, with the application of the brightfield technique as well as with the application of the darkfield technique. This is effected by way of a multitude of individual images, but hereinafter this is represented in each case only by way of one image for reasons of a better overview.

Thus for example, a frequency distribution of the pixel values is determined for the image according to FIG. 2a. The CCD sensor, with which the image according to FIG. 2a has been created comprises a matrix of light-sensitive cells, wherein each cell produces electrical charges corresponding to the fed light quantity, and these charges are outputted as an electrical signal, wherein a pixel value corresponding to the charge value of the cell is determined for each cell. Thus each cell can determine 256 brightness values, from the brightest white to the darkest black, with the resolution of 8 bits which is selected here.

Thereby, for the image according to FIG. 2a it is firstly determined which brightness values, i.e. which pixel values occur in the matrix and in which number. The result is reproduced in the diagram according to FIG. 2b. The pixel value 1 which in the image according to FIG. 2a occurs most often has a brightness of 25 with a frequency of 3.5×105, as is to be recognized in FIG. 2b. Directly adjacent are further brightness values which have a similarly high frequency, and specifically between 20 and 30. The peak 2 of FIG. 2b is arranged in this region and represents the light-grey region of the image according to FIG. 2a. A mean which forms the background signal and represents the bright grey area in FIG. 2a is formed from this peak 2.

This background signal is of a hindrance with the image evaluation, since it glares the entire image. For this reason, it is deducted from each individual pixel value with the later image evaluation, so that an image which in the ideal case has no grey, but a white (actually black) background is provided for the evaluation. This is effected in the reverse manner with the darkfield technique, so that a dark black background is formed in the ideal case, wherein the particles do not distinguish themselves against this black background in a dark color as in FIG. 2a, but in a bright color.

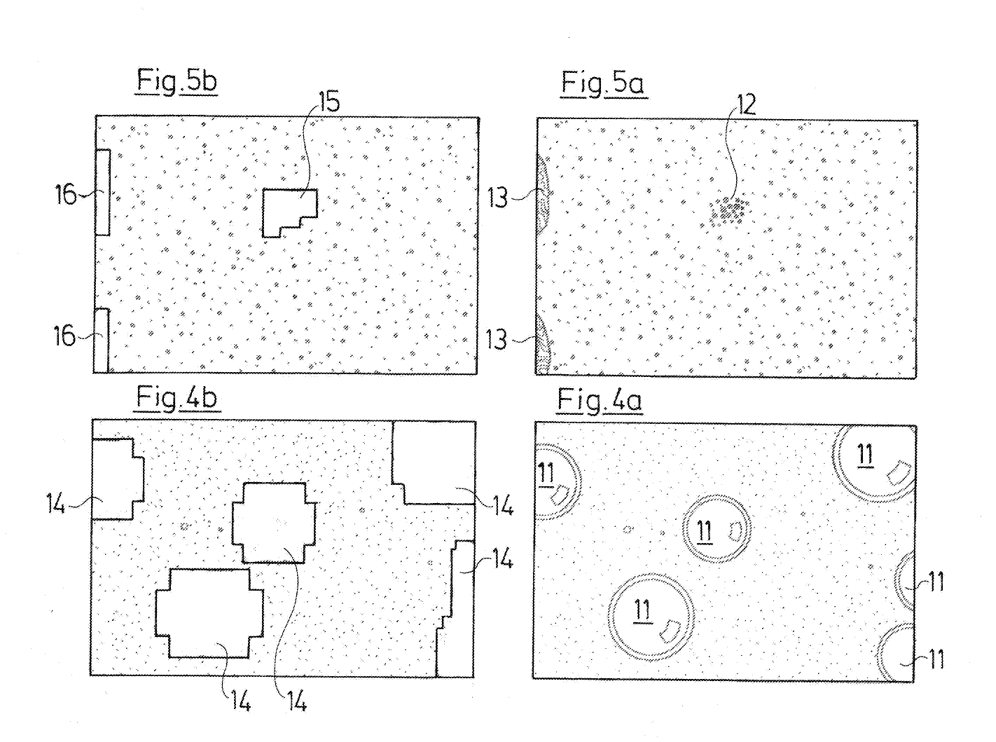

A large dark spot 3 can be recognized in FIG. 2a at the upper left corner of the picture, and this does not affect the background signal since it is comparatively distinctly demarcated. Hereby, it is the case of a spot 3 as is typically produced by an air bubble in the fluid. This dark spot 3 in FIG. 2b is represented by the flat peak 4 next to the peak 2. As can be recognized by way of FIG. 2a as well as by way of the diagram of FIG. 2b, this comparatively distinctly demarcated dark spot 3 has practically no influence on the background signal. Irrespective of this, this region is excluded with the signal evaluation, as will yet be explained further below by way of FIGS. 4 and 5.

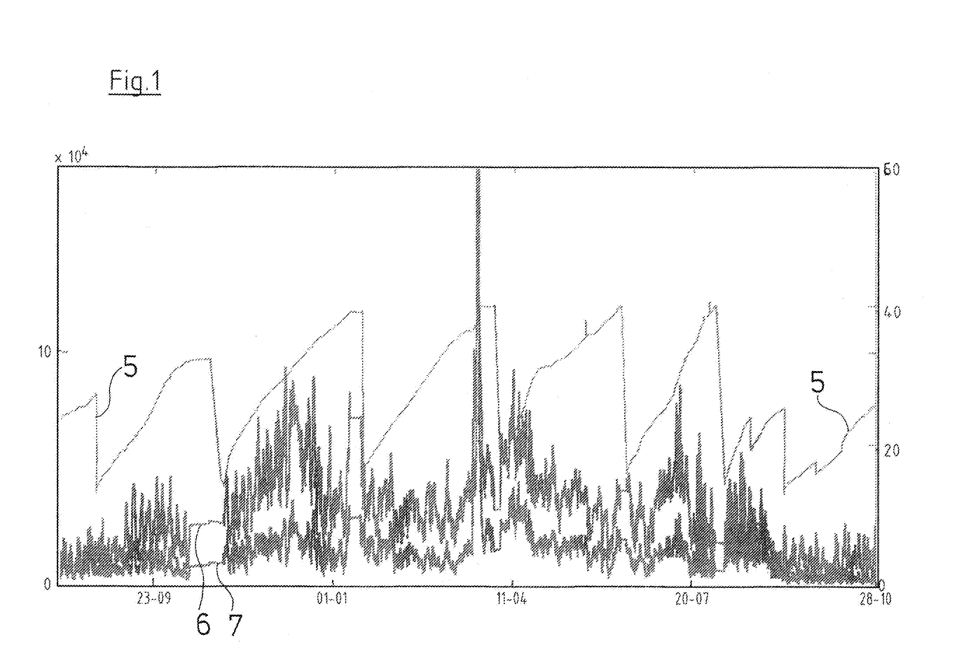

The background signal which can be determined for each image of a fluid sample analyzed by a multitude of images, is formed from a multitude of such images of the same sample, for example as an average value of 500 images, and stored. Thereby, this background signal is not only used for image evaluation, but is also registered over time, as is represented by way of FIG. 1. The temporal course of the background signal is indicated at 5, wherein the horizontal axis indicates the temporal course (in FIG. 1 over roughly one year) and the vertical (right) axis indicates the pixel value, on the basis of which the background signal was formed.

The diagram according to FIG. 1 shows the background signal of a device for monitoring drinking water, with which an exchangeable sample carrier is provided, which, in defined time intervals of e.g. an hour is rinsed with the drinking water to be examined, and then examined in the quasi stationary condition, when the feed and discharge of the sample carrier are closed. Thereby, a biofilm typically is formed at least in the region of the window of the sample carrier in the course of time and this film renders the optical analysis difficult or, from a certain extent, no longer permits the particle detection with the required reliability and accuracy. The sample carrier must then be exchanged or at least cleaned.

The maximal permissible pixel value for the background signal lies at 40 in the diagram according to FIG. 4. The sample carrier must be exchanged or cleaned if this value is reached as a background signal. It is clearly evident from FIG. 1, that this predefined maximal value of 40 has been reached at four locations, whereupon the sample carrier has been exchanged and the background signal has fallen back again below the value 20. These four points in time are roughly at 01-01 (stands for 1st of January), at 01-04 (stands for 1st of April), at 01-06 (stands for 1st June) and at 01-08 (stands for 1st August). The pixel value 40 here represents the predefined limit value for the background signal, with which the method is interrupted, as least the evaluation is interrupted.

Moreover, the speed of the contamination can also be determined by way of the steepness of the curve 5 in the rising regions, and with this, one can reliably predict to a certain extent, as to when the next necessary change of the sample carrier is to be expected. Finally, a predefined limit value can be selected below this upper limit value and this signalizes to the user that the contamination of the window has reached a value which renders an exchange of the sample carrier necessary in the foreseeable future, and thus gives good notice of this.

However, not only the temporal course of the background signal 5 is plotted in FIG. 1, but also the frequency distribution of the detected particles, and specifically in the curve 6, which comprises the particles classified as bacteria, and in the curve 7 which comprises the particles which are classified as non bacteria. As to how such a classification is effected is not the subject matter of the present invention and is therefore not mentioned in more detail. Thereby, although the same horizontal axis as the curve 5 is assigned to these curves 6 and 7, however the vertical axis specifies the frequency distribution on the left scale of the diagram. In particular, as the maximally occurring frequency of 20×104 in the middle of the curve 6 illustrates, this steep increase in the bacteria quantity within the fluid sample accompanies a likewise sharp increase of the background signal, which means of the fouling film in the window which then forms. Otherwise, the diagram however shows that these fluctuations generally have no influence on the coating formation within the window.

FIG. 3a shows a picture, with which the contamination degree of the sample carrier window has significantly increased, and thus the black regions 8 and the streak-like regions 9 are formed by deposits which renders the measurement procedure, which is to say the quantitative and qualitative detection of the particles located in the window, considerably more difficult. As the frequency distribution according to FIG. 3b illustrates, the peak 10 which forms with this image is significantly flatter and wider than the peak 2 which forms with the image according to FIG. 2a. The average value for the background signal and which results from this is accordingly significantly higher, by which means the higher contamination degree is represented.

Not only is the background signal subtracted, but moreover individual groups of cells of the cell matrix are excluded in individual images, on evaluation of the signal, in order to improve the evaluation of the sensor signal. For this, the cell matrix of the sensor which for example can comprise 2560 times 1920 cells is divided into 400 equally large sub-matrices, wherein in the same manner a background signal is formed for each sub-matrix, as was effected in the previously described manner for the complete matrix of cells, for forming the background signal. Again a predefined limit value, specifically a further limit value, which with an 8-bit resolution lies between 0 and 255 and here for example is likewise selected at 40, is set for these sub-matrices. If then sub-matrices result, whose background signal exceeds 40 due to large particles, for example air bubbles 11, accumulations of particles 12 or reflection appearances 13 at the edge, then these are excluded from the further image evaluation, as is represented by way of FIGS. 4b and 5b. Thus there, black, distinctly demarcated fields 14 are to be seen in FIG. 4b, where the air bubbles 11 are to be seen in FIG. 4a, and these fields are formed by a number of such disconnected sub-matrices of cells. Accordingly in FIG. 5b, centrally a field 15 is left out where in FIG. 5a the accumulation of particles 12 is arranged, as well as fields 16 where in FIG. 5a the reflection appearances 13 at the edge are to be seen. A coarse characterization of larger particles with regard to the size can be effected by way of this evaluation. One can moreover ascertain that a further image evaluation is not effected given a maximal number of faded-out fields 14, 15, 16, in order to ensure that the evaluations are always sufficiently statistically reliable.

While specific embodiments of the invention have been shown and described in detail to illustrate the application of the principles of the invention, it will be understood that the invention may be embodied otherwise without departing from such principles.

1. A method for the quantitative and/or qualitative detection of particles in fluid, the method comprising the steps of:
introducing the fluid to be examined into a beam path of an optical device, between at least one light source and the image acquisition sensor with a matrix of light-sensitive cells;
detecting pixel values of the light-sensitive cells and at least partially determining a distribution of the pixel values;
forming with the pixel value or values which have been determined most often a value or average value for a background signal; and
outputting a signal or interrupting the method on reaching a certain limit value for the background signal.

2. A method according to claim 1, wherein a signal is outputted on reaching a first predefined limit value for the background signal and the method is interrupted on reaching a second predefined limit value.

3. A method according to claim 1, wherein the background signal is formed by the mean of values or average values of several images which are recorded one after the other, comprising 500 to 1500 images of different picture planes.

4. A method according to claim 1 wherein the values for the background signal of detections of particles in fluid which are effected temporally one after the other are registered, and speed change is determined by way of the temporal change of the registered values.

5. A method according to claim 1, wherein the values for the background signal of detections of particles in fluid which are effected temporally one after the other are registered, and a determination is made as to when a predefined limit value for the background signal is expected to be reached, by way of the temporal change of the registered values.

6. A method according to claim 1, wherein the background signal is used as a measure for contamination in the beam path and that a fluid carrier receiving the fluid to be examined is cleaned or exchanged on reaching a predefined limit value.

7. A method according to claim 1, wherein the evaluation of the background signal is effected before each detection of particles or at defined temporal intervals.

8. A method according to claim 1, wherein on evaluation of the pixel values, the cell matrix of the sensor is divided into a multitude of part-matrices and a background signal is formed for each part-matrix, and part-matrices with a background signal that exceeds a predefined further limit value, are excluded from the further evaluation for the detection of the particles.

9. A method according to claim 1, wherein the distribution of the pixel values is used to categorize the particles with regard to size.

10. A method according to claim 1, wherein the limit value for the background signal of the cell matrix and/or for the background signal of a part-matrix is defined at 10% to 20% of a maximal pixel value.

11. A method according to claim 1, wherein the limit value for the background signal of the cell matrix given an 8-bit resolution of a pixel value is defined between 30 and 50.

12. A method according to claim 1, wherein the further limit value for the background signal of a part-matrix given an 8-bit resolution is defined between 35 and 45.

13. A device for optical detection of particles in a fluid, the device comprising:
a light source;
an image acquisition sensor having a matrix of light-sensitive cells;
a fluid carrier arranged between the light source and the matrix of light-sensitive cells in a beam path of the light source, and with
control and evaluation electronics which is connected to the image acquisition sensor and is configured to detect pixel values of the cells, at least partly determine a distribution of the pixel values and use the pixel value or values which have been determined most frequently, as a value or average value for a background signal, wherein the control and evaluation electronics are configured to output a signal or for interrupting the detection procedure, on reaching a predefined limit value for the background signal.

14. A device according to claim 13, wherein the control and evaluation electronics are further configured to output a signal on reaching a first predefined limit value for the background signal and interrupt the detecting and determination on reaching a second predefined limit value.

15. A device according to claim 13, wherein the control and evaluation electronics forms the background signal by the mean of values or average values of several images which are recorded one after the other, comprising 500 to 1500 images of different picture planes.

16. A device according to claim 13, wherein the control and evaluation electronics registers the values for the background signal of detections of particles in fluid which are effected temporally one after the other and determines speed change by way of the temporal change of the registered values.

17. A device according to claim 13, wherein the control and evaluation electronics registers values for the background signal of detections of particles in fluid which are effected temporally one after the other, and the control and evaluation electronics makes a determination as to when a predefined limit value for the background signal is expected to be reached, by way of the temporal change of the registered values.

18. A device according to claim 13, wherein the control and evaluation electronics uses the background signal as a measure for contamination in the beam path and provides an indication that a fluid carrier receiving the fluid to be examined is to be cleaned or exchanged on reaching a predefined limit value.

19. A device according to claim 13, wherein the control and evaluation electronics effects an evaluation of the background signal before each detection of particles or at defined temporal intervals.

20. A device according to claim 13, wherein the control and evaluation electronics, on evaluation of the pixel values, divides the cell matrix of the sensor into a multitude of part-matrices and a background signal is formed for each part-matrix, and part-matrices with a background signal that exceeds a predefined further limit value, are excluded from the further evaluation for the detection of the particles.
